(19) United States
(12) Patent Application Publication
(10) Pub. No.: US 2013/0220328 A1
(43) Pub. Date: Aug. 29, 2013

(54) GAS DELIVERY CONDUIT MANAGEMENT SYSTEM

(71) Applicant: Gregory John Jablonski, Eindhoven (NL)

(72) Inventor: Gregory John Jablonski, Eindhoven (NL)

(73) Assignee: KONINKLIJKE PHILIPS ELECTRONICS N.V., Eindhoven (NL)

(21) Appl. No.: 13/882,305

(22) PCT Filed: Oct. 25, 2011
(86) PCT No.: PCT/IB2011/054748

§ 371 Date: Apr. 29, 2013

Related U.S. Application Data
(60) Provisional application No. 61/408,804, filed on Nov. 1, 2010.

(51) Int. Cl. A61M 16/08; A61M 16/06; F16L 3/02; A61M 16/00

(52) U.S. Cl. CPC A61M 16/0875; A61M 16/0057; A61M 16/06; F16L 3/02
USPC 128/205.25; 248/75

(57) ABSTRACT

An apparatus (22) for managing a gas delivery conduit (14) of a respiratory therapy includes a socket member (28) having a main body portion (32) and a first coupling portion (40). The main body portion is structured to receive and hold a portion of the gas delivery conduit in a manner such that the gas delivery conduit slides along the main body portion. Main body portion include an attachment member having clip member (46) and a second coupling portion (48). The socket member and the attachment member attach to one another through coupling of the first coupling portion and the second coupling portion. The socket member and the attachment member rotate relative to one another when the socket member and the attachment member are attached.

This patent application claims the priority benefit under 35 U.S.C. §119(e) of U.S. Provisional Application No. 61/408,804 filed on Nov. 1, 2010, the contents of which are herein incorporated by reference.

### BACKGROUND OF THE INVENTION

1. Field of the Invention

The present invention relates to respiratory therapy systems, such as non-invasive ventilation and pressure support systems, and, in particular, to an apparatus for managing a gas delivery conduit in a respiratory therapy system.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Patients that must utilize the respiratory therapies as just described are often confronted with the problem of managing the tubing between the ventilator or pressure support device and the patient interface device, e.g. preventing the tubing from becoming in the patient's bedding. The length of this tubing is typically 6 feet. In particular, they frequently struggle with tubing torque on the patient interface device, and overall tubing positioning as body movement occurs while asleep. These issue often frustrate the patient and can interfere with the effective delivery of therapy.

### SUMMARY OF THE INVENTION

Accordingly, it is an object of the present invention to provide a gas delivery conduit management system for use in a respiratory therapy system that address the shortcomings of conventional respiratory therapy systems. This object is achieved according to one embodiment of the present invention by providing a gas delivery conduit management system that includes a socket member having a main body portion and a first coupling portion. The main body portion receives and holds a portion of the gas delivery conduit in a manner such that the gas delivery conduit is able to slide along the main body portion. The main body portion also includes an attachment member having clip member and a second coupling portion. The socket member and the attachment member are selectively attachable to one another through coupling of the first coupling portion and the second coupling portion. The socket member and the attachment member are able to rotate relative to one another when the socket member and the attachment member are attached to one another through coupling of the first coupling portion and the second coupling portion. In this manner the main body portion holds the gas delivery conduit at a relatively fixed location during use of the respiratory therapy system.

In another embodiment, a respiratory therapy system is provided that includes a pressure generating device, a gas delivery conduit coupled to the pressure generating device, a patient interface device coupled to the gas delivery conduit, and an apparatus for managing the gas delivery conduit as just described.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2 is a schematic diagram of a portion of the system shown in FIG. 1 showing a tubing management apparatus according to an exemplary embodiment of the invention forming a part the system of FIG. 1;

FIG. 3 is a right side elevational view and FIG. 4 is a front elevational view of a socket member of the tubing management apparatus shown in FIGS. 1 and 2 according to an exemplary embodiment of the invention;

FIG. 5 is a side elevational view and FIG. 6 is a bottom plan view of an attachment member of the tubing management apparatus shown in FIGS. 1 and 2 according to an exemplary embodiment of the invention; and

FIG. 7 is a side elevational view showing socket member of FIGS. 3 and 4 coupled to the attachment member of FIGS. 5 and 6 to form the tubing management apparatus shown in FIGS. 1 and 2.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

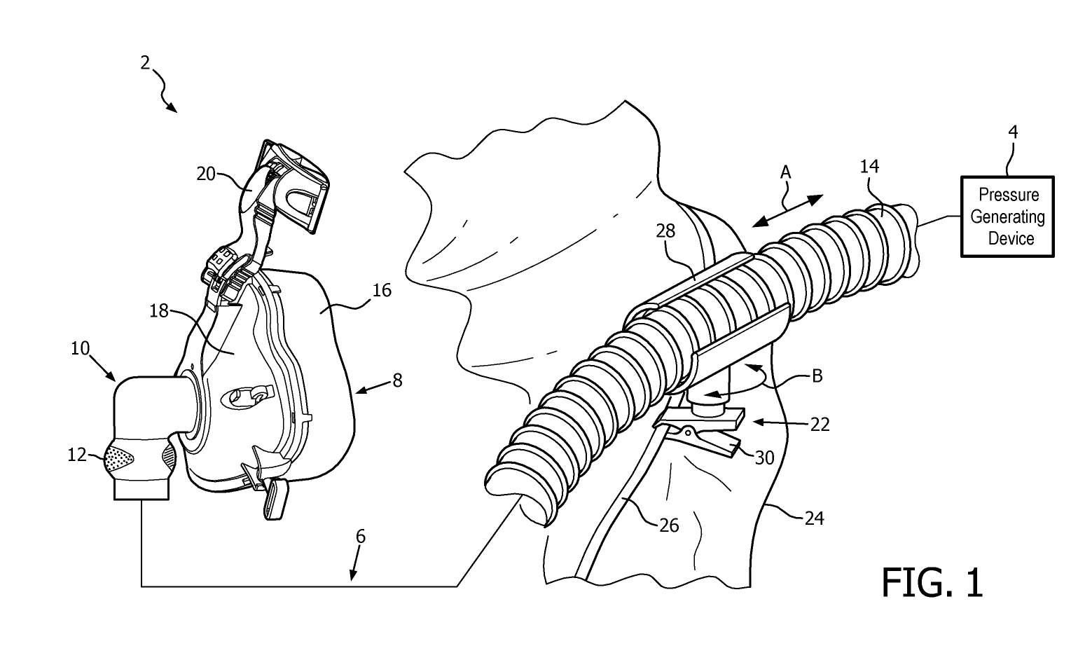

A system 2 adapted to provide a regimen of respiratory therapy to a patient according to one exemplary embodiment is generally shown in FIG. 1. System 2 includes a pressure generating device 4, a patient circuit 6, a patient interface device 8, and an elbow conduit 10 having an exhaust port 12 provide therein. Although system 2 is discussed as including pressure generating device 4, patient circuit 6, patient interface device 8, and elbow conduit 10, it is contemplated that other systems may be employed while remaining within the scope of the present invention. For example, and without limitation, a system in which pressure generating device 4 is coupled to a patient interface device having an integrated exhaust port assembly is contemplated.

Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Patient circuit 6 is structured to communicate the flow of breathing gas from pressure generating device 4 to patient interface device 8, and, as shown in FIG. 1, includes a gas delivery conduit or tube 14.

Patient interface 8 is typically a nasal or nasal/oral mask structured to be placed on and/or over the face of a patient. Any type of patient interface device 8, however, which facilitates the delivery of the flow of breathing gas to, and the removal of a flow of exhalation gas from, the airway of such a patient may be used while remaining within the scope of the present invention. In the embodiment shown in FIG. 1, patient interface 8 includes a cushion 16, a rigid shell 18, and a forehead support 20. Straps (not shown) of a headgear component may be attached to shell 18 and forehead support 20 to secure patient interface 8 to the patient's head. An opening in shell 18 to which elbow conduit 10 is coupled allows the flow of breathing gas from pressure generating device 4 to be communicated to an interior space defined by shell 18 and cushion 16, and then, to the airway of a patient. The opening in shell 18 also allows the flow of exhalation gas (from the airway of such a patient) to be communicated to exhaust port 12 of elbow conduit 10 in the current embodiment.

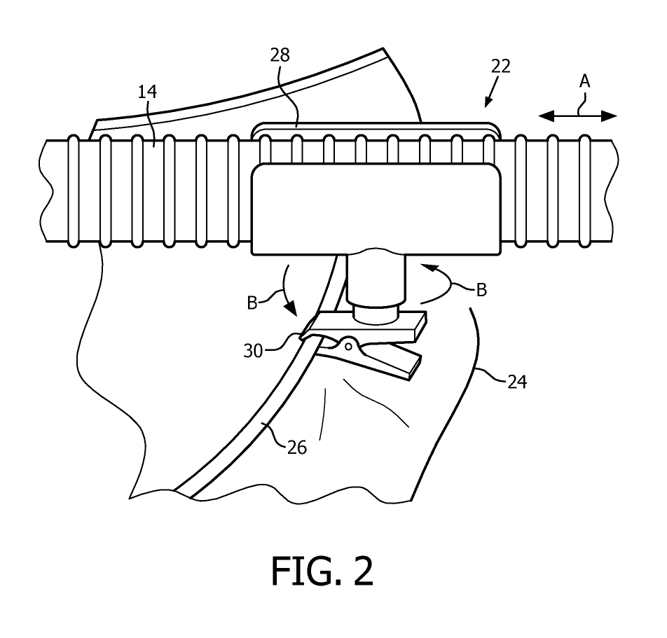

As seen in FIGS. 1 and 2, system 2 further includes tubing management apparatus 22, which, as described in greater detail herein, is structured to be selectively attached to a mattress 24 (or another part of the patient bed or bedding) and provide for the management of gas delivery conduit 14. In the illustrated embodiment, tubing management apparatus 22 is structured to be selectively attached to a bed seam 26 of mattress 24, although other connection configurations and locations are also possible. Tubing management apparatus 22 includes a socket member 28 and an attachment member 30.

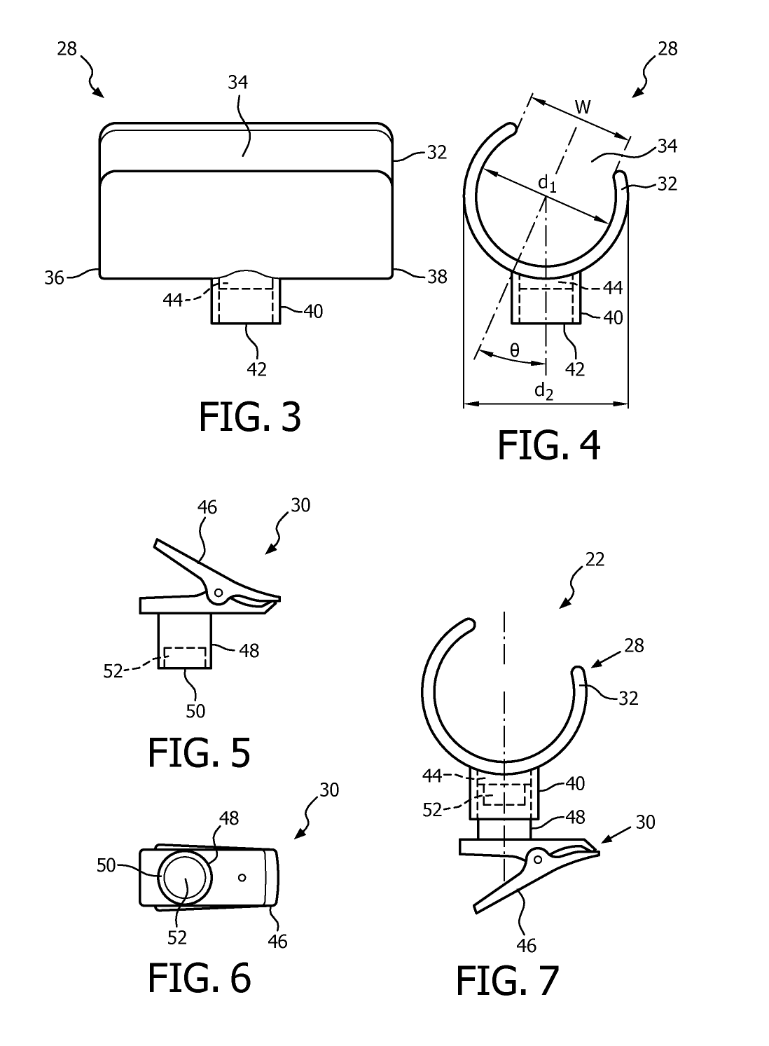

FIG. 3 is a right side elevational view and FIG. 4 is a front elevational view of socket member 28 according to an exemplary embodiment of the invention. Socket member 28 includes an elongated, arcuate-shaped main body portion 32 defining an opening 34 along the length thereof In an exemplary embodiment, socket member 28 is formed from a rigid material. However, the present invention contemplates that the socket member can be formed from a semi-rigid or flexible material so long as it is sufficiently rigid to provide support for and hold the gas delivery conduit.

As seen in FIG. 3, the bottom of the front end 36 and the rear end 38 of main body portion 32 flair outwardly. The significance of this outward flaring is described elsewhere herein. In the exemplary embodiment, the length of main body portion 32 is 2.100 inches, the inner diameter d₁ of main body portion 32 is 1.115 inches and the outer diameter d₂ of main body portion 32 is 1.225 inches. In an alternative embodiment the length of main body portion 32 may be between 1.500 inches and 2.500 inches, d₁ may be between 1.000 inches and 1.250 inches and d₂ may be between 1.100 inches and 1.400 inches. In addition, in the exemplary embodiment, the length l of opening 34 is 0.800 inches. In an alternative embodiment, the width w of opening 34 may be between 0.750 inches and 0.900 inches.

Socket member 28 also includes a post member 40 extending downwardly from the bottom of main body portion 32. As seen in FIG. 4, in the exemplary embodiment, the longitudinal axis of post member 40 is offset from the centerline of main body portion 32 passing through opening 34 and dividing of main body portion 32 into two symmetrical halves by an angle θ equal to between 20 and 25 degrees. In the exemplary embodiment, post member 40 is 0.415 inches long and has a bore 42 provided therein that is 0.517 inches in diameter and 0.415 inches in depth at its deepest point (outer edges), although other dimensions are possible. A magnet 44 is provided within bore 42 (sitting at 0.276 inches depth in the exemplary embodiment). In the exemplary embodiment, magnet 44 is 0.500 inches in diameter and is 0.125 inches thick.

FIG. 5 is a side elevational view and FIG. 6 is a bottom plan view of attachment member 30. Attachment member 30 includes clip member 46 and post member 48 extending downwardly from the bottom of clip member 46. In the exemplary embodiment, post member 48 is 0.340 inches long, 0.500 in diameter and has a bore 50 provided therein that is 0.380 inches in diameter and 0.125 inches in depth, although other dimensions are possible. A magnet 52 is provided within bore 50. In the exemplary embodiment, magnet 52 is 0.375 inches in diameter and is 0.125 inches thick. The polarity of magnet 52 is opposite the polarity of magnet 44.

In operation, attachment member 30 is coupled to socket member 28 to form tubing management apparatus 22 by inserting post member 48 of attachment member 30 into post member 40 (into the bore 42 thereof) of socket member 28 as shown in FIG. 7. The coupling in this manner thus self locating. When this is done, magnet 44 and magnet 44 and magnet 52 will be attracted to one another and attachment member 30 and socket member 28 will be held together. Attachment member 30 and socket member 28 will, however, be able to rotate relative to one another about the longitudinal axis through each post member 40, 48.

Tubing management apparatus 22 may then be attached to bed seam 26 of mattress 24, and gas delivery conduit 14 may be inserted within main body portion 32 through opening 34. When this is done, gas delivery conduit 14 is free to move (slide) within main body portion 32 in the direction of arrows A shown in FIGS. 1 and 2 (the flaring of the ends 36, 38 facilitates this sliding), and socket member 28 is free to rotate in the direction of arrows B shown in FIGS. 1 and 2 to provide for the management of gas delivery conduit 14 for better positioning (e.g., during sleep) and to lessen torque thereon while a patient is receiving respiratory treatment using patient interface device 8 and pressure generating device 4. Tubing management apparatus 22 also prevents gas delivery conduit 14 from bending or binding while movement occurs.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An apparatus for managing a gas delivery conduit of a respiratory therapy system, comprising:
a socket member having a main body portion and a first coupling portion, the main body portion being structured to receive and hold a portion of such a gas delivery conduit in a manner wherein the gas delivery conduit is able to slide along the main body portion; and
an attachment member having a clip member and a second coupling portion, wherein the first coupling :portion comprises a first post member extending from the main body portion and having a first bore, wherein a first magnet is positioned within the first bore, wherein the second coupling portion comprises a second post member extending from the clip member and having a second bore, wherein a second magnet is positioned within the second bore, wherein the socket member and the attachment member are selectively attachable to one another through coupling of the first post member to the second post member and magnetic attraction between the first magnet and the second magnet resulting from coupling of the first post member to the second post member, and wherein the socket member and the attachment member are able to rotate relative to one another responsive to the socket member and the attachment member being attached to one another through coupling of the first post member to the second post member.

2. (canceled)

3. The apparatus according to claim 1, wherein the first post member and the second post member are selectively attachable to one another by inserting the second post member into the first bore.

4. The apparatus according to claim 3, wherein the first post member is 0.415 inches long, wherein the first bore has a diameter of 0.517 inches and a maximum depth of 0.415 inches, wherein the second post member is 0.340 inches long and 0.500 in diameter, and wherein the second bore is 0.380 inches in diameter and 0.125 inches in depth.

5. The apparatus according to claim 3, wherein the main body portion is arcuate-shaped and defines an opening along a length thereof

6. The apparatus according to claim 5, wherein a longitudinal axis of the first post member is offset from a centerline of the main body portion passing through the opening and dividing of the main body portion into two symmetrical halves by an angle θ.

7. (canceled)

8. The apparatus according to claim 5, wherein an inner diameter of the main body portion is 1.115 inches and an outer diameter of the main body portion is 1.225 inches.

9. The apparatus according to claim 5, wherein an inner diameter of the main body portion is between 1.000 inches and 1.250 inches and an outer diameter of the main body portion is between 1.100 inches and 1.400 inches.

10. The apparatus according to claim 5, wherein a width of the opening is 0.800 inches.

11. The apparatus according to claim 5, wherein a width of the opening is between 0.750 inches and 0.900 inches.

12. The apparatus according to claim 1, wherein the main body portion is arcuate-shaped and defines an opening along a length thereof, wherein a bottom of a front end and a bottom of a rear end of the main body portion each flair outwardly.

13. (canceled)

14. The apparatus according to claim 10, wherein a length of the main body portion is between 1.500 inches and 2.500 inches.

15. A respiratory therapy system, comprising:
a pressure generating device for generating a flow of breathing gas
a gas delivery conduit coupled to the pressure generating device;
a patient interface device coupled to the gas delivery conduit; and
an apparatus for managing a gas delivery conduit including:
a socket member having a main body portion and a first coupling portion, the main body portion being structured to receive and hold a portion of the gas delivery conduit in a manner wherein the gas delivery conduit is able to slide along the main body portion; and
an attachment member having a clip member and a second coupling portion, therein the first coupling portion comprises a first post member extending from the main body portion and having a first bore, wherein a first magnet is positioned within the first bore, wherein the second coupling portion comprises a second post member extending from the clip member and having a second bore, wherein a second magnet is positioned within the second bore, wherein the socket member and the attachment member are selectively attachable to one another through coupling of the first post member to the second post member and magnetic attraction between the first magnet and the second magnet resulting from coupling of the first post member to the second post member, and wherein the socket member and the attachment member are able to rotate relative to one another when the socket member and the attachment member are attached to one another through coupling of the first post member to the second post member.

16. (canceled)

17. The respiratory therapy system according to claim 15, wherein the first post member and the second post member are selectively attachable to one another by inserting the second post member into the first bore.

18. The respiratory therapy system according to claim 17, wherein the first post member is 0.415 inches long, wherein the first bore has a diameter of 0.517 inches and a maximum depth of 0.415 inches, wherein the second post member is 0.340 inches long and 0.500 in diameter, and wherein the second bore is 0.380 inches in diameter and 0.125 inches in depth.

19. The respiratory therapy system according to claim 17, wherein the main body portion is arcuate-shaped and defines an opening along a length thereof.

20. The respiratory therapy system according to claim 19, wherein a longitudinal axis of the first post member is offset from a centerline of the main body portion passing through the opening and dividing of the main body portion into two symmetrical halves by an angle θ.

21. (canceled)

22. The respiratory therapy system according to claim 19, wherein an inner diameter of the main body portion is 1.115 inches and an outer diameter of the main body portion is 1.225 inches.

23. The respiratory therapy system according to claim 19, wherein an inner diameter of the main body portion is between 1.000 inches and 1.250 inches and an outer diameter of the main body portion is between 1.100 inches and 1.400 inches.

24. The respiratory therapy system according to claim 19, wherein a width of the opening is 0.800 inches.

25. The respiratory therapy system according to claim 19, wherein a width of the opening is between 0.750 inches and 0.900 inches.

26. The respiratory therapy system according to claim 15, wherein the main body portion is arcuate-shaped and defines an opening along a length thereof, wherein a bottom of a front end and a bottom of a rear end of the main body portion each flair outwardly.

27. (canceled)

28. (canceled)
